(19) United States
(12) Patent Application Publication
(10) Pub. No.: US 2011/0070126 A1
(43) Pub. Date: Mar. 24, 2011

(54) UNIT FOR THE LOADING AND IDENTIFICATION OF BIOLOGICAL SAMPLES AND INTEGRATED MACHINE COMPRISING SAID LOADING AND IDENTIFICATION UNIT

(75) Inventor: Paolo Galiano, Padova (IT)

(73) Assignee: ALIFAX SPA CON SOCIO UNICO, Polverara (IT)

(21) Appl. No.: 12/994,576

(22) PCT Filed: May 26, 2009
(86) PCT No.: PCT/EP2009/056354

§ 371 Date: Nov. 24, 2010

(30) Foreign Application Priority Data
May 27, 2008 (IT) UD2008A000128

(51) Int. Cl. G01N 33/50

(52) U.S. Cl. 422/64; 422/67

(57) ABSTRACT

A unit for the loading and identification of different biological samples, liquid, semi-liquid or mixed, to be subjected to one or more different analyses of chemical-biological type, such as immunological or coagulative reactions, or chemical-physical type, comprising: a plurality of support members able to carry a plurality of test tubes each one containing a determinate biological sample to be analyzed; a plurality of identification means based on optical or optoelectronic recognition, which carry information, univocal for a determinate sample or a determinate group of samples to be analyzed and which are directly associated with each of the test tubes and/or with each of the support members so that said information is transmitted to an electronic processing unit for the selection of a determinate test tube depending on the type of biological sample and the analysis to be carried out; a pick-up member able to pick up, on the basis of determinate commands received from the electronic processing unit, a portion of the sample contained in the determinate test tube and to dispense and move the portion of the sample to send the portion of the sample to the reactions provided in a reaction unit and to the subsequent analytical measurements in a measuring unit.

### FIELD OF THE INVENTION

The present invention concerns a machine provided with a unit for loading and identifying biological samples and able to analyze, with random sample loading or batch loading, different types of analysis on different types of biological sample, such as blood, serum, urine and other biological fluids. In particular the machine according to the present invention is usable to measure the presence, even quantitative, of analytes in biological samples of different types, assessing the progress of suitable reactions, such as, for example the immunological, agglutination/aggregation type, coagulation or chemical or other type.

### BACKGROUND OF THE INVENTION

The flow of work in a modern laboratory uses automatic instruments (cell-sorters) which prepare specific containers, according to the type of sample or anticoagulant contained in the test tubes, to be sent to the specific dedicated instrument or dedicated reaction unit, which does the analysis.

Therefore, there are dedicated analytical machines downstream of said cell-sorter which receive samples of whole blood or, after centrifugation, samples of plasma or serum or urine etc., and subject them to the desired reactions, propedeutic to the determinate analysis.

Laboratories that are not supplied with said cell-sorters separate and sort the test tubes manually, and load them manually into the analytical machines dedicated to this type of sample.

Typically, in a laboratory dedicated to carrying out several types of analyses on different biological samples, such as blood, serum, urine, feces etc, as many analysis stations are provided as there are various types of analyses to be carried out. Each analysis station is equipped with a dedicated analytical machine, while a conveyor belt, fed from the cell-sorter, is provided to sequentially move the various samples in the test tube between the various stations, where samples are taken or not depending on the type of analysis to be carried out on the specific sample. In fact, nowadays, analytical laboratory instruments carry out tests on pre-selected types of samples.

All this is disadvantageous, particularly in a laboratory that analyzes different types of biological sample in order to effect different types of reaction for various measurements of a chemical-biological and chemical-physical type: it has the disadvantage that each analytical machine, with its relative dedicated machine, occupies a determined bulk and physical space, has a determinate procedure for use, requires determinate and specific reagents, and occupies a determinate number of operators. As is obvious, this leads to a high waste of resources in terms of space, time, resources, raw materials and economic cost.

One purpose of the present invention is to achieve a loading and identification unit of biological samples to be analyzed and an integrated analytical machine to carry out one or more types of analysis on several different biological samples comprising said loading and identification unit, which allows to integrate the possibility of carrying out one or more analyses and reactions on the same samples taken from the patient which requires analysis on serum or blood or whatever combination is required, in random or batch type execution, or different biological samples taken in random or batch type tests.

Another purpose is to achieve a machine which is both compact and automatically manages the load and the recognition of a determinate biological sample and the subsequent determination and execution of the type of analysis required, thus determining a reduction in the space required to do said analyses, a more practical use, a saving in time and costs of the operators required to carry out the analyses.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

According to a first feature of the present invention, a loading and identification unit of different biological samples, liquid, semi-liquid or mixed, such as: blood, liquids of the blood (plasma, serum), cerebrospinal fluid, pleural fluid, peritoneal fluid, pericardial fluid, amniotic fluid, seminal fluid, biological secretions of the physiological channels (vaginal, ocular, . . . ), urine, feces dissolved in liquid and other is used to load an integrated analytical machine in which one or more analyses of chemical-biological type are carried out, such as immunological or coagulative reactions, of the chemical-physical type on several types of different biological samples.

The loading and identification unit comprises:

a plurality of support members able to carry a plurality of test tubes each one containing a determinate biological sample to be analyzed;

a plurality of identification means based on optical or optoelectronic recognition, which carry univocal recognition information for a determinate sample or for a determinate group of samples to be analyzed and which are directly associated with each of the test tubes and/or each of the support members, so that this information is transmitted to an electronic processing unit for the selection of a determinate test tube depending on the type of biological sample and analysis to be carried out;

a pick-up member able to pick up, according to determinate commands received from the electronic processing unit, a portion of the sample contained in the determinate test tube and to move said portion of sample to send it to the reactions provided in a reaction unit and to the subsequent analytical measurements in a measuring unit.

The loading and identification unit allows to load, both randomly, and according to determinate grouping criteria, test tubes containing biological samples even different from each other, and to identify the contents of the test tubes automatically and with certainty. On the basis of the identification, which is transmitted to the electronic processing unit, the loading and identification unit also picks up the determinate test tube and sends it to reaction and analysis. It is then the reaction unit and the unit that measures the reaction, suitably instructed on the type of sample, that will carry out the suitable reactions and analyses.

According to one embodiment of the present invention, the loading and identification unit comprises a reading device based on optical or optoelectronic reading, able to acquire the information of the identification means and to transmit this information to the electronic processing unit for the selection of a determinate test tube depending on the type of biological sample and the analysis to be carried out.

Alternatively it is the operator who visually identifies the samples and consequently configures the functioning of the loading and identification unit and subsequently the reaction and measuring unit.

According to one form of embodiment of the present invention, the identification means comprise bar codes or RFID tags applied to each of the test tubes and/or to each of the support members, generally called racks.

According to a variant embodiment, each of the support members, or racks, is colored with a determinate color which serves as an identification means of the chromatic type (either automatically or by the operator) associated with the type of biological sample contained in the test tubes carried by the individual support member, identifiable, for example, by a specific bar code. The operator prepares racks for the samples of blood, serum or diluted feces or that which he thinks is most opportune according to the type of sample pre-selected in the containing rack provided and which is to be inserted in the analytical instrument.

According to a second feature of the present invention, an integrated analytical machine to carry out one or more types of analysis on several types of different biological samples comprises:

a unit to analyze biological samples, comprising a reaction unit able to carry out a desired reaction depending on the determinate type of biological sample to be analyzed and a measuring unit to carry out a determinate analytical measurement on the reacted sample depending on the determinate type of biological sample;

a unit to manage the reagents, which manages the reagents required by the reaction unit depending on the type of analysis to be carried out and the type of sample to be reacted and analyzed;

an electronic processing unit able to control at least the analysis unit and the reagent management unit.

The analysis unit comprises a loading and identification unit comprising:

a plurality of support members able to carry a plurality of test tubes each containing a determinate biological sample to be analyzed;

a plurality of identification means based on optical or optoelectronic recognition, which carry information, univocal for a determinate sample or a determinate group of samples to be analyzed and which are directly associated with each of the test tubes and/or with each of the support members or racks, so that this information is transmitted to an electronic processing unit for the selection of a determinate test tube depending on the type of biological sample and analysis to be carried out.

a pick-up member able to pick up, advantageously through a needle, according to determinate commands received from the electronic processing unit, a portion of the sample contained in the determinate test tube and to move and dispense said portion of sample to send it to the reactions provided in the reaction unit and to the subsequent analytical measurements in the measuring unit.

Advantageously the pick-up member is also able to carry out an auto-wash, between the taking of one sample and another, so as to avoid carrying over and contamination, which is important when different biological samples to be analyzed are taken.

The loading and identification unit for the biological samples to be analyzed and the relative integrated analytical machine allow to integrate the possibility of carrying out one or more analyses and reactions on several types of different biological samples, and of automatically managing the loading and the recognition of a determinate biological sample and the subsequent determination and execution of the type of analysis required. There is, in fact, an automatic recognition of the type of sample contained in the test tubes and on the basis of this recognition it is possible to automatically instruct the analytical machine to carry out the desired and necessary reactions and analyses. The resulting analytical machine is very compact, thus determining a reduction in the spaces required for carrying out said analyses, a more practical use and a saving in time and costs of the operators responsible for carrying out the analyses.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a schematic representation of an analytical machine according to the present invention;

FIG. 2 is a perspective view of a part of the machine in FIG. 1;

FIG. 3 is a front view of FIG. 2;

FIG. 4 is a lateral view of FIG. 2;

FIG. 5 is a schematic representation of a part of the machine in FIG. 1.

### DETAILED DESCRIPTION OF A PREFERENTIAL FORM OF EMBODIMENT

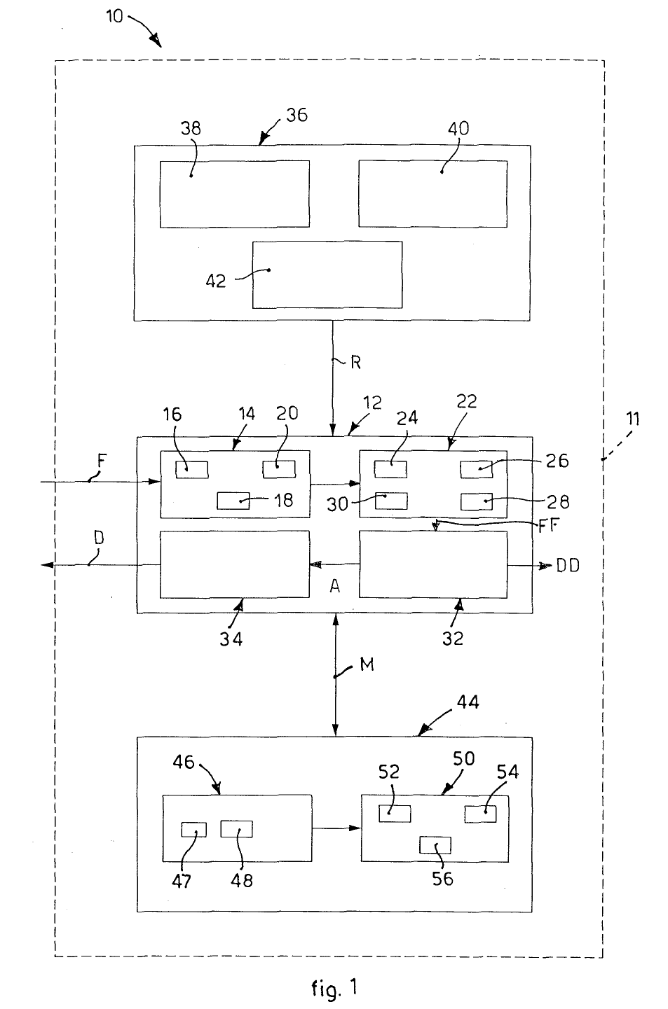

With reference to FIG. 1, an integrated analytical machine 10 to perform one or more types of analysis on several types of different biological sample provides a box-like container 11 of the compact type in which the following groups of components are integrated:

a unit 12 to analyze the biological sample;

a management unit 36 to manage the reagents needed for the analyses, by means of which the various reagents required are managed according to the type of analysis to be performed and the type of sample to be reacted and analyzed. The reagents normally consist of a suspension of covered nanospheres, of polystyrene or latexes, or with a colloidal gold base, sensitized with a specific antigen/antibody;

an electronic processing unit 44 to process the result of the measurement, to control the analysis unit 12 and the management unit 36 and advantageously also to render an intelligible and interpretable result of the analysis carried out available to an operator.

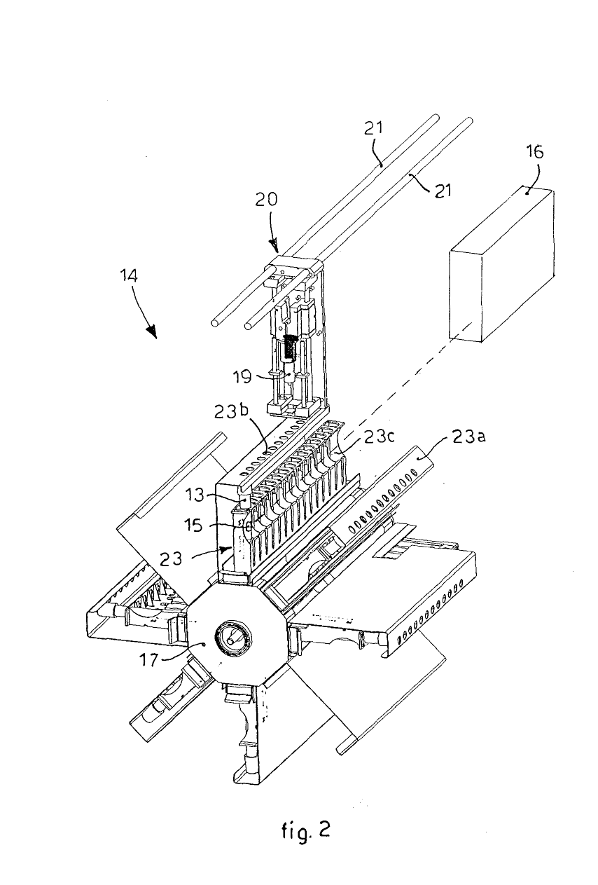

The analysis unit 12 comprises a loading and identification unit 14 (FIGS. 2, 3 and 4) for the biological samples to be analyzed, in order to load a plurality of test tubes 13, suitably sealed, for example with a stopper 13a of the type that can be perforated, containing the various types of biological samples to be reacted and analyzed.

The loading and identification unit 14 comprises a plurality of racks 23, mounted on a rotary drum 17, each of which is able to carry the test tubes 13 in relative seatings 23c.

The test tubes 13 are kept in their seatings in the rack 23 thanks to lids 23a, suitably shaped, which hold them and prevent them from falling.

The loading and identification unit 14 exploits determinate identification devices 15, typically based on optical or optoelectronic recognition, to recognize the type of sample contained in each test tube 13 and other connected information, or in a group of test tubes 13 supported by a determinate rack 23.

In this way, the test tubes 13 can be loaded randomly in the racks 23, or according to determinate criteria for loading and grouping. This is possible because, in any case, the loading and identification unit 14 will recognize them automatically.

For example, each test tube 13 can be provided with an identification device 15, such as a determinate bar code, or RFID tag, or other of an optoelectronic type.

Information on the type of biological sample contained is memorized in the identification device 15, and possibly the type of analysis to be carried out, plus possible data relating to the patent from whom the sample has been taken. This is convenient when the test tubes 13 are loaded randomly, without any criterion, and therefore the content of one is not correlated to the content of the other test tubes 13 in the same rack 23 or other racks.

It may also be that each rack 23 is made with a predefined color (which functions as a reading device), and univocally associated with the type of biological sample that is contained in the test tubes 13 that it supports (chromatic recognition). For example, each color identifies the origin of the biological sample; blood, urine, feces, other biological fluids.

Otherwise, or in addition, each rack 23 can also have its own identification device 15, such as a bar code or RFID tag or other of an optoelectronic type. The solutions where the rack 23 is identified by a precise color or by a bar code or RFID tag are useful when the test tubes 13 are loaded on each rack 23 in a group having common properties (same type of sample, same analysis to be done or other).

Advantageously, it may also be that both each test tube 13 and also each rack 23 is provided with said identification devices 15, in the form of a bar code or RFID tag.

In any case, a reading device 16 is provided, of the type with optical or optoelectronic reading which, according to one or the other of the solutions described above, it is able to recognize the color of one or the other of the racks 23, or to read the content of the determinate bar code or RFID tag associated with each of the test tubes 13 and/or the rack 23.

The reading device 16 can be used manually by the operator, or it can be integrated with an automatism into the loading and identification unit 14. It may also be that, in the solution of chromatic recognition, the identification of the color is made visually by an operator who consequently instructs the machine 10.

In this case, an aperture may be provided in the container 11 of the machine 10 exactly in correspondence with one of the racks 23, so as to be able to effect manually the optical scanning and reading of the information on the test tubes, or to allow an operator to recognize visually, by means of the determinate coloring, the type of biological sample.

The reading device 16 transmits the information detected to an information acquisition unit 46; this, by means of a microprocessor 48, is able to associate the information read or detected by the racks 23 and/or the test tubes 13, with the determinate analyses to be carried out on the sample: this means both the type of analysis and the characteristic parameters for the various analytical procedures (typically time, temperature, reaction kinetics), the type and quantity of reagents, all information pre-memorized or memorizable according to an input, even temporarily, in an electronic memory 47.

The acquisition unit 46 can be connected to a remote server from which, by possibly correlating an identification of the patient to the information associated with the sample to be analyzed, it can take the correct instructions on which reaction and analysis to carry out on the sample.

According to these instructions, the microprocessor 48 is able to execute determinate reaction programs pre-loaded in the memory 47; the programs are able to be selected by the operator, by means of a user interface, such as a keyboard and display, according to the sample to be reacted and the type of reaction, and therefore analysis, to which the sample is to be submitted.

In the loading and identification unit 14 a suction and movement device 20 is provided, having a syringe 19 with a needle, in order to take in part of the sample from a selected test tube 13.

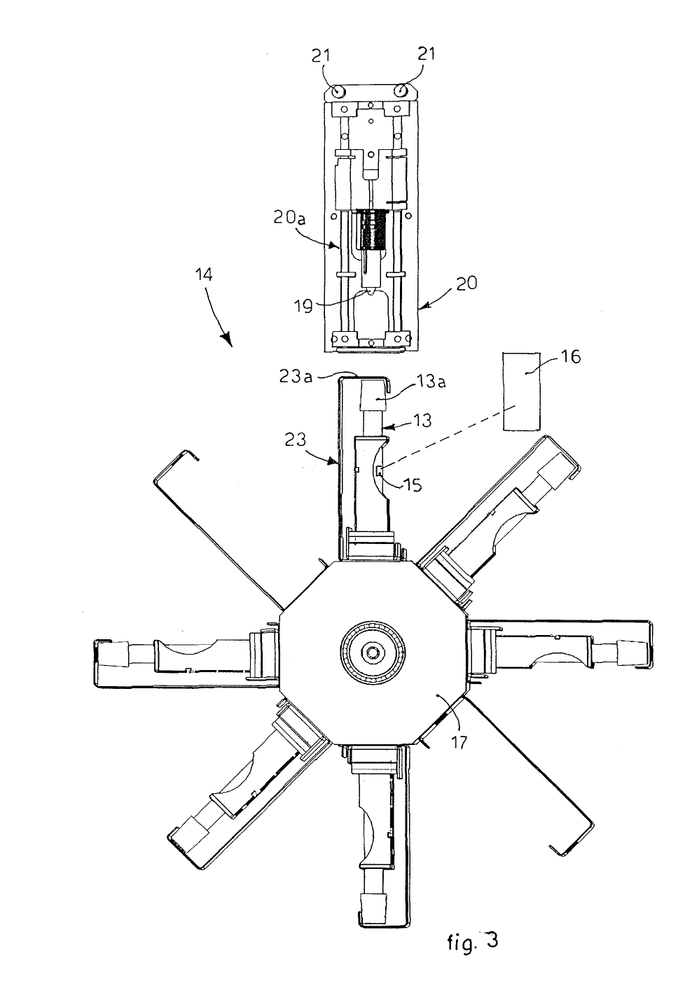

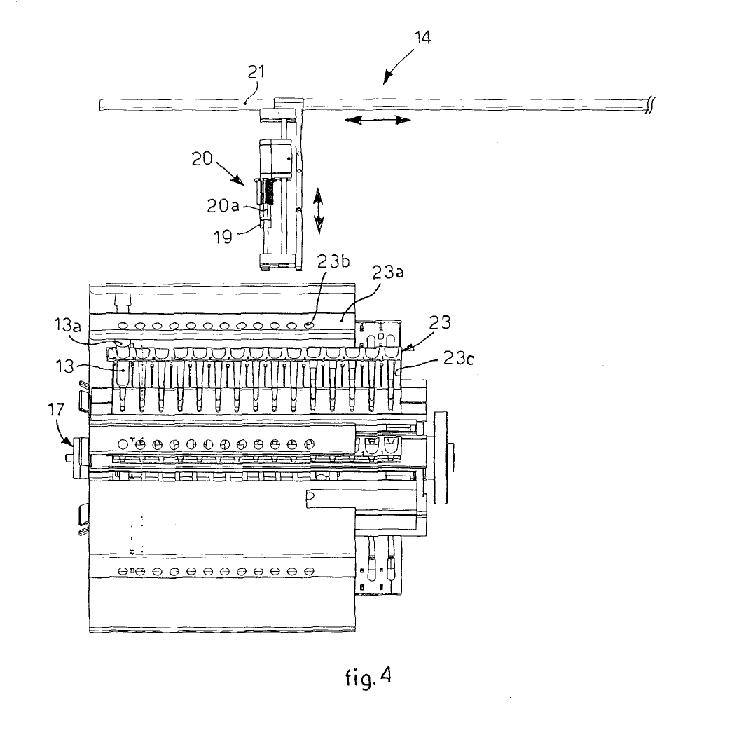

The suction and movement device 20 is able to be translated automatically and commanded by the microprocessor 48, according to the test tube 13 from which the sample is to be taken. Translation is possible both horizontally along guides 21, and also vertically, by means of precision linear actuators 20a (FIG. 3), all driven by drive means, not shown in the drawings, controlled by the microprocessor 48. In this way the syringe 19 interacts with the test tube 13 selected, passing through a hole 23b (FIG. 4) made in the lid 23a of the racks 23, in correspondence with each test tube 13 and subsequently perforating the stopper 13a in order to take the sample.

If the test tubes 13 are loaded randomly, without any precise order, the reading device 16 reads the information on the first test tube 13 available. Once the information associated with the test tube 13 has been read, the microprocessor 48 commands the syringe 19 to move along the guides 21, to the position where the desired test tube 13 is found from which the sample to be analyzed is to be taken: it then commands the syringe 19 to descend, to take in the sample and to return to position. At this point, the machine 10 has all the information necessary on the sample taken, and also a portion of the sample, and can continue with the desired analyses.

When the analyses of the test tubes 13 in a determinate rack 23 have been terminated, the drum 27 rotates and makes available a new group of test tubes 13 of a new rack 23.

Or it may be that the test tubes 13 are loaded in groups according to predetermined criteria (for example by type of sample or analysis) on different racks 23 each identified by a bar code or a color, which is read or recognized and acquired by the microprocessor 48.

In this case, having this information, the selection of the desired rack 23 and the determinate test tube 13 to be analyzed is made by the microprocessor 48 according to the predetermined work programs loaded in its memory 47.

Consequently, the microprocessor 48 commands the rotary drum 17 to rotate, so that the desired rack 23 is disposed in the angular position suitable to cooperate with the syringe 19 (FIG. 2) and then the samples are taken from the various test tubes 13.

The loading and identification unit 14, in which the samples remain for a determinate period of time between loading and sampling, is advantageously provided with a thermostat device 18, to preserve perishable samples correctly, preferably between about 4° C. and 8° C.

The analysis unit 12 provides a pre-analysis unit 22, by means of which it carries out the necessary pre-analysis operations on the sample taken in by the syringe 19, which operations are propedeutical to the correct reaction and measuring of the sample. In particular, the suction and movement device 20 can carry the sample to a mixer/homogenizer device 24 and/or to a filtration device 26, and/or to a centrifugation device 28, with relative dilutions and/or sedimentations, according to needs. For example, a sample of whole blood could be centrifuged to separate the plasma from the corpuscle part.

Advantageously, the suction and movement device 20 is also able to auto-wash inside the syringe 19, so as to prevent any carry over effects between one sampling and the next.

After this, a sampling member 30 picks up a portion of the pre-treated sample and transmits it to a reaction device 32. The sampling member 30 can also transmit other portions of the sample to other machines or analytical instruments, outside the machine 10.

The reaction device 32 can be configured, automatically or by the operator, to effect different types of reactions on different types of sample, by means of determinate reagents taken from the reagent management unit 36, all based on the recognition of the sample carried out by the loading and identification unit 14.

The management unit 36 comprises a heat preservation unit 38, in which the reagents are contained in suitable tanks and are preserved at a temperature comprised between about 4° C. and 8° C., controlled by a thermostat 42. The reagents, which are substantially suspended, have to be remixed before they are dispensed, and therefore a mixing unit 40 is provided, suitable for this purpose.

According to one solution of the invention, the reaction device 32 is made as described in the Italian patent application, in the name of the present Applicant, filed at the same time as the present application and incorporated herein as a reference.

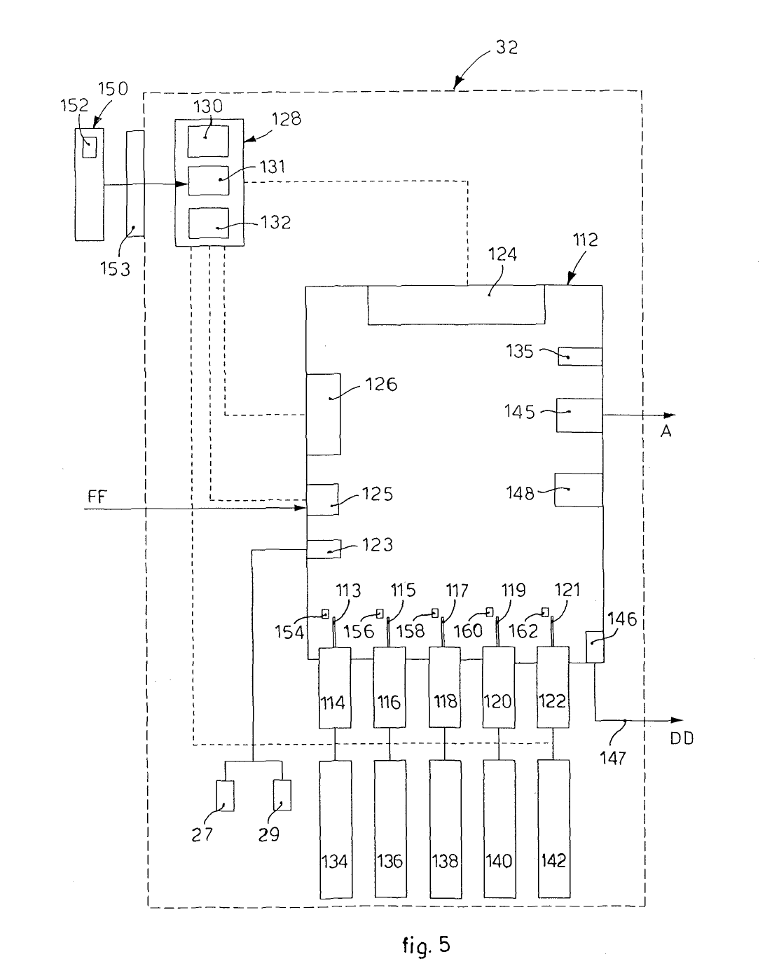

In particular, the reaction device 32 is provided with a reaction cell 112 into which, in sequence, a plurality of biological samples, liquid, semi-liquid or mixed, of analogous or different types, are subjected to a reaction for a determinate analysis, and comprises (FIG. 5):

a first dispenser element 125 able to introduce (as indicated by the arrow FF) a predetermined quantity of the biological sample inside the cell 112. The quantity derives directly from the sampling member 30;

a plurality of microvalve dispensers 114, 116, 118, 120, 122, each of which is able to nebulize a determinate microvolume of a desired reagent inside the cell 112, wherein the type of reagent and the amount of the microvolume of reagent are chosen as a function of the specific reaction to be carried out, selected from a group comprising: reactions for analyses of a chemical-biological type, such as immunological or coagulative reactions, or of a chemical-physical type;

a mixer element 124 which is able to mix the biological sample with the selected reagent;

a second dispenser element 145 able to send to analytical measurement a determinate quantity of the biological sample after reaction with the selected reagent;

washing means 148 and discharge means 146, 147, able respectively to wash the inside of the cell 112, between one reaction and the other, and to discharge the content of the cell 112 (as indicated by the arrow DD), so as to prepare the cell 112 for a subsequent reaction;

electronic processing means 128 able to command and control, in the desired times and modes, at least the functioning of both the first dispenser element 125 which dispenses the sample, of the microvalve dispensers 114, 116, 118, 120, 122 which dispense the reagents, and of the mixer element 124, and also of the second dispenser element 145 which sends the reacted sample for measurement, and of the washing means 148 and discharge means 146, 47, as a function of the type of reaction to be performed and the type of biological sample to be analyzed, so that their functioning is synchronized and automated according to predetermined memorized reaction programs, selectable by an operator.

The electronic processing means 128 comprise an electronic memory 130 (it may consist of the same memory 47 in FIG. 1) in which the desired reaction programs have been memorized and a microprocessor 131 able to execute the programs (which can also be the same microprocessor 48 in FIG. 1, suitably configured).

The microprocessor 131, according to a selected work program, is able to transmit other command signals at least to activate/de-activate the functioning and/or regulate the quantities dispensed by the first 125 and the second 145 dispenser element and by the microvalve dispensers 114, 116, 118, 120, 122.

The micro-dispensation of each of the nozzles 113, 115, 117, 118 and 121 of the microvalve dispensers 114, 116, 118, 120 and 122 is accurately controlled by respective photometers 154, 156, 158, 160, 162, which substantially count the drops actually delivered.

The microprocessor 131, according to a selected work program, is able to transmit command signals to activate/de-activate the functioning and/or regulate the mixing power of the mixer element 124.

The electronic processing means 128 also comprise a timer 132 by means of which the microprocessor 131 is able to control and synchronize the functioning times both of the first dispenser element 125 which dispenses the sample, the microvalve dispensers 114, 116, 118, 120, 122 which dispense the reagents and the mixer element 124, and also the second dispenser element 145 which sends the reacted sample for measurement, and also of the washing means 148 and the discharge means 146, 147.

The reaction unit 32 can comprise heating means 126 able to heat and thermostat the inside of the cell 112 to a desired temperature value, correlated to the type of reaction to be made and according to a control signal received from the electronic processing means 128.

Blowing means 135 can also be provided, able to blow in a selected fluid inside the cell 112, according to the type of reaction to be made and according to a control signal received from the electronic processing means 128.

The reaction unit 32 is associated with a plurality of tanks 134, 136, 138, 140, 142, which were mentioned previously with regard to the management unit 36, to contain a determinate reagent, each of which is connected to a corresponding microvalve dispenser 114, 116, 118, 120, 122. The tanks 134, 136, 138, 140, 142 can be managed directly by said reagent management unit 36.

It is also possible to have means to control the pressure 123, able to control and selectively vary the pressure inside the cell 112, according to the type of reaction to be made and according to a control signal received from the electronic processing means 128. The pressure control means 123 cooperate, under the control of the processing means 128, with a pump 127 or an aspirator 129, in order to selectively put the inside of the cell 112 under pressure or vacuum, according to the reaction needs.

A preferred embodiment provides that the machine 10, and the reaction device 32, only function by means of an electronic card 150, which is associated with a specific production batch of reagents, having electronic or magnetic means 152, for example a magnetic strip or microchip, at least with memorization capacities, in which data are memorized for the use of the reaction device. The data are read by reading means 153 and transmitted to the processing means 128, and possibly memorized, even only temporarily, in the electronic memory 130. In particular, said data comprise at least a univocal identification associated with the determinate and specific production batch of reagents; the reading by the reading means 153 and the recognition of the univocal identification by the processing means 128 providing the consent to use the reaction device by an operator.

Furthermore, it may also be that in the electronic or magnetic means 152 data are also memorized relating to the correct and necessary microvolumes to be dispensed for the reagent of the specific production batch of reagents, and all the reaction parameters. In particular, apart from the times and temperatures of the reactions, the specific calibration curve may be memorized for each production batch in order to execute every individual reaction and analysis, without any further wastage of reagent for executing said analyses, or using a single point on the curve as a reference of good reliability of the production batch of reagents.

The electronic or magnetic means 152 function only for each production batch of reagents with which an identification code is associated for the correct execution of the test, and are not re-usable.

Said data are acquired by reading the reading means 153 by the processing means 128, which according to the data acquired are thus able to command at least the correct dispensing of the microvolumes of the reagents by the microvalve dispensers 114, 116, 118, 120, 122.

The use of the card 150 is particularly advantageous in conjunction with the reagent management unit 36, which can be suitably instructed, thanks to the data contained in the electronic or magnetic means 152, also regarding the methods of preserving the reagents themselves.

According to a variant, particularly advantageous in antigen-antibody reactions, the mixer element 124 is the adaptive type according to the concentration and mass to be mixed, as described in the Italian patent application in the name of the present Applicant filed at the same time as the present application and incorporated here as a reference.

The adaptive mixer element 124 provides a piezoelectric actuator that exerts a mechanical wave on the content of the chamber 112, the frequency of oscillation of which is modified by a microprocessor, for example the processing means 128.

This is possible thanks to a closed control ring provided with a sensor of piezoelectric force which detects the intensity of the mechanical wave generated by the actuator and which passes through the chamber 112. In this way we have a modulation of the frequency used in mixing, also depending on the changes in density which are obtained by the mixing action itself. The system can also be applied to the mixer/homogenizer 24 of the pre-analysis unit 22.

Once the desired reaction has been carried out, part of the reacted sample is thus transmitted to a measuring unit 34, of a known type, or in any case able to be selected from measuring units of a known type, according to the type of measurement that is to be carried out. It registers the reaction phenomenon, kinetic or at the final point, using techniques of absorption of electromagnetic, acoustic or heat radiations.

Once the measurement has been made, the part of the sample analyzed is discharged, as indicated by arrow D.

The result of the measurement is transmitted to the processing unit 44 which, by means of the microprocessor 48, processes the result and transmits it to a reporting unit 50, which makes it available to monitors 52 and for a printer 54. The result, in the desired visual format, can be memorized in a memory 56, for future consultations and reference.

It is clear that modifications and/or additions of parts may be made to the unit for the loading and identification of biological samples to be analyzed, and integrated analytical machine to carry out one or more types of analysis on several types of different biological samples comprising said loading and identification unit as described heretofore, without departing from the field and scope of the present invention.

It is also clear that, although the present invention has been described with reference to some specific examples, a person of skill in the art shall certainly be able to achieve many other equivalent forms of unit for the loading and identification of biological samples to be analyzed, and integrated analytical machine to carry out one or more types of analysis on several types of different biological samples comprising said loading and identification unit, having the characteristics as set forth in the claims and hence all coming within the field of protection defined thereby.

1. A unit for the loading and identification of different biological samples, liquid, semi-liquid or mixed, to be subjected to one or more different analyses of chemical-biological type, such as immunological or coagulative reactions, or chemical-physical type, the unit comprising:
a plurality of support members able to carry a plurality of test tubes each one containing a determinate biological sample to be analyzed;
a plurality of identification means based on optical or optoelectronic recognition, which carry information, univocal for a determinate sample or a determinate group of samples to be analyzed and which are directly associated with each of the test tubes and/or with each of the support members so that said information is transmitted to an electronic processing unit for the selection of a determinate test tube depending on the type of biological sample and the analysis to be carried out;
a pick-up member able to pick up, on the basis of determinate commands received from the electronic processing unit, a portion of the sample contained in the determinate test tube and to dispense and move said portion of the sample to send said portion of the sample to the reactions provided in a reaction unit and to the subsequent analytical measurements in a measuring unit.

2. The unit as in claim 1, wherein the identification means comprise bar codes or RFID tags applied on each of the test tubes and/or on each of the support members.

3. The unit as in claim 1, wherein each of the support members is colored a determinate color that functions as an identification mean of the chromatic type associated with the type of biological sample contained in the test tubes carried by the individual support member.

4. The unit as in claim 1, comprising a reading device based on optical or optoelectronic reading, able to acquire the information from the identification means and to transmit the information to the electronic processing unit in order to select a determinate test tube according to the type of biological sample and the analysis to be done.

5. The unit as in claim 1, wherein the support members are able to be moved, according to a command transmitted by the electronic processing unit, in order to make selectively available to the pick-up member the desired test tube and/or the desired group of test tubes contained in a determinate support member.

6. The unit as in claim 1, wherein the support members comprise a plurality of seatings to house the test tubes in predetermined positions, separated from each other.

7. The unit as in claim 1, wherein the support members comprise holding means able to hold the loaded test tubes in position.

8. The unit as in claim 7, wherein the holding means are shaped like a folded wall that has an upper hole in correspondence with the position of each test tube, for the passage of the pick-up member.

9. The unit as in claim 1, wherein the support members comprise racks mounted on a rotary drum.

10. The unit as in claim 1, wherein the pick-up member comprises a suction element to pick up the portion of sample to be analyzed.

11. The unit as in claim 10, wherein the suction element is mounted sliding on horizontal guides to be selectively translated between the support members and the reaction unit.

12. The unit as in claim 10, wherein the pick-up member comprises linear actuator means to lift/lower the suction element with respect to the support members.

13. The unit as in claim 10, wherein the pick-up member is configured to effect an auto-washing at least of the suction element, between picking up one sample and the other, so as to prevent any carry-over effect.

14. The unit as in claim 1, comprising thermostat means.

15. An integrated analytical machine to carry out one or more types of analysis on several types of different biological sample, comprising:
an analysis unit to analyze the biological sample, comprising a reaction unit able to carry out a desired reaction depending on the determinate type of biological sample to be analyzed and a measuring unit to carry out a determinate analytical measurement on the reacted sample depending on the determinate type of biological sample;
a management unit to manage the reagents, by means of which the various reagents required by the reaction unit are managed according to the type of sample to be reacted and analyzed;
an electronic processing unit able to control at least the analysis unit and the reagent management unit;
wherein the analysis unit comprises a loading and identification unit comprising:
a plurality of support members able to carry a plurality of test tubes, each containing a determinate biological sample to be analyzed;
a plurality of identification means based on optical or optoelectronic recognition, carrying information, univocal for a determinate sample or a determinate group of samples to be analyzed and which are directly associated with each of the test tubes and/or with each of the support members, so that said information is transmitted to an electronic processing unit for the selection of a determinate test tube according to the type of biological sample and analysis to be done;
a pick-up member able to pick up, according to determinate commands received from the electronic processing unit, a portion of the sample contained in the determinate test tube and to move said portion of sample so as to send said portion of sample to the reactions provided in the reaction unit and the subsequent analytical measurements in the measuring unit.

16. The machine as in claim 15, wherein the electronic processing unit comprises an acquisition unit provided with a microprocessor, which receives and processes both the information from the reading device and also the results of the measurement done by the measuring unit, and with electronic memorization means in which data correlated with the analyses to be made on the determinate biological samples identified can be registered.

17. The machine as in claim 16, wherein the loading and identification unit comprises a reading device based on optical or optoelectronic reading, able to acquire the information from the identification means and to transmit the information to the electronic processing unit in order to select a determinate test tube according to the type of biological sample and the analysis to be done.

18. The machine as in claim 15, wherein the electronic processing unit comprises a reporting unit provided with display means, to render visually available the results of the analysis processed by the microprocessor, and memorization means to memorize said results.

19. The machine as in claim 15, wherein said reagents consist of a suspension of coated nanospheres, of polystyrene or latexes, or with a colloidal gold base, sensitized with a specific antigen/antibody or chemical reagents in liquid phase.

20. The machine as in claim 15, wherein the management unit is able to manage tanks containing said reagents, mixing the content thereof by means of dedicated mixing members, and/or providing for their heat stability by means of heat preservation means and thermostat means.

21. The machine as in claim 15, wherein the measuring unit is able to measure the reaction phenomenon, kinetic or at the final point, using techniques of absorption of electromagnetic, acoustic or heat radiations.

22. The machine as in claim 15, further comprising a pre-analysis unit, downstream of the loading and identification unit, which is provided with pre-treatment means for the biological sample picked up, and a sampling member able to pick up a portion of the pre-treated sample in order to send said portion of the pre-treated sample to the reaction unit.

23. The machine as in claim 15, wherein the reaction device is provided with a reaction cell into which, in sequence, the biological sample, liquid, semi-liquid or mixed, of analogous or different types, are subjected to a reaction for a determinate analysis, said reaction unit comprising:
a first dispenser element able to introduce a predetermined quantity of the biological sample inside the cell;
a plurality of microvalve dispensers, each of which is able to nebulize a determinate microvolume of a desired reagent inside the cell, wherein the type of reagent and the amount of the microvolume of reagent are chosen as a function of the specific reaction to be carried out, selected from a group comprising: reactions for analyses of a chemical-biological type, such as immunological or coagulative reactions, or of a chemical-physical type;
a mixer element which is able to mix the biological sample with the selected reagent;
a second dispenser element able to send to analytical measurement a determinate quantity of the biological sample after reaction with the selected reagent;
washing means and discharge means, able respectively to wash the inside of the cell, between one reaction and the other, and to discharge the content of the cell so as to prepare the cell for a subsequent reaction;
electronic processing means able to command and control, in the desired times and modes, at least the functioning of both the first dispenser element which dispenses the sample, of the microvalve dispensers which dispense the reagents, and of the mixer element, and also of the second dispenser element which sends the reacted sample to the measuring unit, and of the washing means and the discharge means, as a function of the type of reaction to be performed and the type of biological sample to be analyzed, so that their functioning is synchronized and automated according to predetermined memorized reaction programs, selectable by an operator.

24. The machine as in claim 15, comprising an electronic card, which is associated with a specific production batch of reagents, having electronic or magnetic means, at least with memorization capacities, in which data are memorized for the use of the reaction device, said data being read by reading means and transmitted to the processing means.

25. The machine as in claim 24, further comprising a microprocessor, wherein the processing means and the memorization means of the reaction unit consist respectively of the microprocessor itself and electronic memorization means of the acquisition unit.
